(19) United States
(12) Patent Application Publication
(10) Pub. No.: US 2022/0096788 A1
(43) Pub. Date: Mar. 31, 2022

(54) PACKAGING FOR MEDICAL DEVICES

(71) Applicant: BOSTON SCIENTIFIC SCIMED, INC., Maple Grove, MN (US)

(72) Inventor: AJAY GUPTA, SHOREVIEW, MN (US)

(73) Assignee: BOSTON SCIENTIFIC SCIMED, INC., MAPLE GROVE, MN (US)

(21) Appl. No.: 17/547,480

(22) Filed: Dec. 10, 2021

Related U.S. Application Data
(63) Continuation of application No. PCT/US2020/037228, filed on Jun. 11, 2020.
(60) Provisional application No. 62/860,758, filed on Jun. 12, 2019.

(51) Int. Cl. A61M 25/00

(52) U.S. Cl. CPC A61M 25/002; A61M 2205/0216

(57) ABSTRACT

Medical device packages are disclosed. An example package may include an elongate carrier tube capable of having a medical device disposed therein. The carrier tube may include an elastomeric material. The carrier tube may include a tube wall and a slit may be formed in the tube wall.

### CROSS-REFERENCE TO RELATED APPLICATIONS

This application This application is a continuation of International Application No. PCT/US2020/37228, filed Jun. 11, 2020, which claims the benefit of priority under 35 U.S.C. § 119 to U.S. Provisional Application Ser. No. 62/860,758 filed Jun. 12, 2019, the entirety of which is incorporated herein by reference.

### TECHNICAL FIELD

The present disclosure pertains to medical devices, and methods for manufacturing medical devices. More particularly, the present disclosure pertains to packaging for medical devices.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. A package for a medical device is disclosed. The package comprises: an elongate carrier tube capable of having a medical device disposed therein; wherein the carrier tube includes an elastomeric material; and wherein the carrier tube includes a tube wall and wherein a slit is formed in the tube wall.

Alternatively or additionally to any of the embodiments above, the tube wall is substantially transparent so that the medical device can be visualized within the carrier tube.

Alternatively or additionally to any of the embodiments above, the elastomeric material includes silicone.

Alternatively or additionally to any of the embodiments above, the elastomeric material includes ethylene propylene diene monomer rubber, ethylene propylene rubber, polyethylene terephthalate glycol, polytetrafluoroethylene, foamed polytetrafluoroethylene, ethylene vinyl acetate, an ionomer, a fluoropolymer, or combinations thereof.

Alternatively or additionally to any of the embodiments above, the slit extends axially along the tube wall.

Alternatively or additionally to any of the embodiments above, the slit includes one or more undulations.

Alternatively or additionally to any of the embodiments above, the carrier tube is capable of be arranged in a spiral configuration.

Alternatively or additionally to any of the embodiments above, when the carrier tube is in the spiral configuration, the carrier tube includes a plurality of adjacent loops that are secured together by one or more discrete bonding members.

Alternatively or additionally to any of the embodiments above, at least some of the one or more discrete bonding members includes an adhesive.

Alternatively or additionally to any of the embodiments above, the carrier tube is capable of be arranged in a helical configuration.

A method for packaging a medical device is disclosed. The method comprises: extruding a carrier tube; wherein the carrier tube includes an elastomeric material; wherein the carrier tube includes a tube wall; forming a slit in the tube wall; and loading a medical device into the carrier tube by passing the medical device through the slit.

Alternatively or additionally to any of the embodiments above, the tube wall is substantially transparent, and further comprising inspecting the medical device by viewing the medical device through the tube wall.

Alternatively or additionally to any of the embodiments above, forming a slit in the tube wall includes forming an axial slit in the tube wall.

Alternatively or additionally to any of the embodiments above, forming a slit in the tube wall includes forming an undulating slit in the tube wall.

Alternatively or additionally to any of the embodiments above, further comprising arranging the carrier tube in a spiral configuration.

Alternatively or additionally to any of the embodiments above, when the carrier tube is in the spiral configuration the carrier tube includes a plurality of adjacent loops, and further comprising securing together at least some of the plurality of adjacent loops with one or more discrete bonding members.

Alternatively or additionally to any of the embodiments above, at least some of the one or more discrete bonding members includes an adhesive.

Alternatively or additionally to any of the embodiments above, further comprising arranging the carrier tube in a helical configuration.

A carrier tube for packaging a medical device is disclosed. The carrier tube comprises: an extruded silicone tube having a tube wall; wherein the extruded silicone tube is substantially transparent; and a slit formed in the tube wall.

Alternatively or additionally to any of the embodiments above, the slit includes an axial region, an undulating region, or combinations thereof.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a perspective view of an example medical device package.

FIG. 2 is a perspective view of a portion of an example medical device package.

FIG. 3 is a perspective view of a portion of an example medical device package.

FIG. 4 is a top/side view of a portion of an example medical device package.

FIG. 5 is a top/side view of a portion of an example medical device package.

FIG. 6 schematically depicts an example arrangement/configuration for a medical device package.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

Elongated medical devices may be contained within appropriate packaging that allows the medical devices to be protected during shipping, storage, preparation, and/or usage. For example, prior to sale, elongated medical devices such as guidewires, catheters, and the like, may be sterilized and packaged. When suitably packaged, the elongated medical devices can be transported to an appropriate medical setting where they can be stored until needed for use. Because devices may be susceptible to damage when shipped and/or stored, some or all of the device may be disposed within a tube or shell often termed a carrier tube. The carrier tube may be part of the packaging that affords the device some level of protection during the shipping/storage phase as well as during preparation or use prior to or during a clinical intervention. Other desirable benefits may also be achieved through the use of a carrier tube as part of the packaging. For example, the carrier tube may be coiled (e.g., arranged in a spiral configuration), and maintain the elongated medical device in a configuration that may be easier and/or more convenient to store. Further, a carrier tube may help protect the elongated device during removal of the device from other packaging (e.g. box or pouch, etc.). Additionally, the use of a carrier tube may aid a user to better maintain the sterility and control of the device during an operation.

Disclosed herein are medical device packages. The packages may include a number of desirable features and benefits. For example, the packages may include materials that allow for a desirable amount of flexibility, transparency, utility, and/or other features. Other features and advantages may also be appreciated, as disclosed herein.

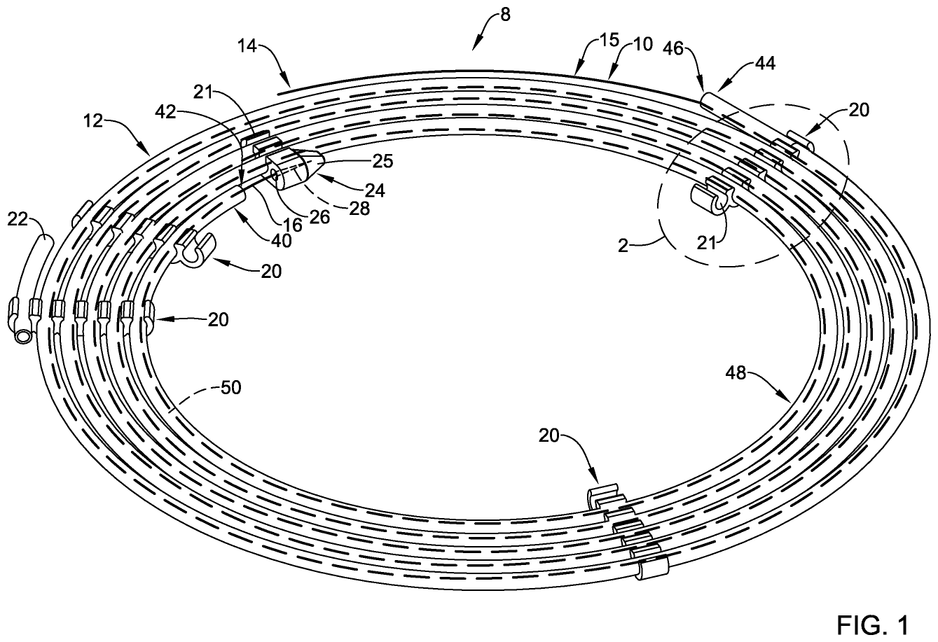

FIG. 1 illustrates an example embodiment, showing a perspective view of a medical device 10 disposed in a packaging assembly 8 including a carrier tube 12 and a securement member 24. In this example, the medical device 10 may be a guidewire. In other embodiments, medical device 10 may be another type of elongated medical device, such as a catheter or the like. The elongated medical device 10 includes a first end portion 16 and a second end portion 14, and a body portion 15 disposed between the two end portions. In FIG. 1, a large part of the body portion 15 is disposed within, and thereby covered by, the carrier tube 12. In some embodiments, only a portion of the body 15 is disposed within the carrier tube 12, while in other embodiments the entire body portion 15 may be disposed within the carrier tube 12. In some embodiments, the first end portion 16 may be a proximal end portion and may include the proximal most terminus of the medical device 10, and the second end portion 14 may be a distal end portion and may include the distal-most terminus of the medical device 10. In other embodiments, this may be reversed (e.g., the first end portion may be the proximal portion of the medical device and the second end portion may be the distal portion).

The carrier tube 12 includes a first end 40 (e.g., a distal end 40) with a first opening 42, a second end 44 (e.g., a proximal end 44) with a second opening 46, and a tube wall 48 defining a lumen 50 extending between the first opening 42 and the second opening 46. The carrier tube 12 is configured to receive a portion of the elongated medical device 10 within the lumen 50. For example, as shown in FIG. 1, the body portion 15 is disposed within the lumen 50 of the carrier tube 12.

The carrier tube 12 may generally be configured to hold the medical device 10 in a suitable configuration. In at least some embodiments, the carrier tube 12 may be arranged or otherwise configured as a spiral or “coiled” configuration (e.g., as depicted in FIG. 1) that allows the medical device 10, which may have a reasonably long length, to be held in a compact configuration. The individual windings of the spiral/coil may be secured together by one or more clips 20. For example, each of the clips 20 may include two or more clip portions 21, each clip portion 21 configured to selectively and/or releasably engage or grip a portion of the carrier tube 12 to hold it in a desired configuration (e.g., a spiral configuration, a helical configuration, etc.). Optionally, a carrier tube stub portion 22, which may simply be a shortened section of tube connected to one of the outer clips 20, may also be included. Such a portion 22 in combination with the carrier tube 12, may be used, for example, in maintaining control of an end portion of the medical device, while allowing a section of the medical device to be exposed for manipulation by a user. Other configurations for the carrier tube 12 include configurations suitable for holding other medical devices, as desired.

The packaging assembly may include a securement member 24 coupled and/or attached to the carrier tube 12 for selectively and/or releasably securing the medical device 10 within and/or relative to the carrier tube 12. The securement member 24 may include a body portion 25. The body portion 25 may define an opening 26 that provides a pathway into a tapered cavity and/or channel 28 defined by the body portion 25. The channel 28 may be configured to receive an end portion (e.g., the first end portion 16) of the medical device 10, and secure the end portion of the medical device 10 therein—thereby securing the medical device 10 relative to the carrier tube 12.

The carrier tube 12 may be formed from a suitable material. In some instances, the carrier tube 12 may include an elastomeric material. For example, the carrier tube 12 may include silicone (e.g., the carrier tube 12 may comprise an extruded silicone tube). Other materials are contemplated including ethylene propylene diene monomer rubber, ethylene propylene rubber, polyethylene terephthalate glycol, polytetrafluoroethylene, foamed polytetrafluoroethylene, ethylene vinyl acetate, ionomers, fluoropolymers, and/or the like. When using such materials, the carrier tube 12 may have a relative high level of flexibility. For example, the carrier tube 12 may be about 1.5-20 times more flexible than standard carrier tubes, or about 1.5-10 times more flexible than standard carrier tubes, or about 1.75-10 times more flexible than standard carrier tube, or about 2-10 times more flexible than standard carrier tubes, or about 2-5 times more flexible than standard carrier tubes. This allows the carrier tube 12 to be arranged in a variety of shapes including spiral shapes, helical shapes, non-spiral/helical shapes, stacked shapes, geometric shapes, irregular shapes, and/or the like. This may include the carrier tube 12 taking the shape of the medical device 10 disclosed therein. For example, in instances where the medical device 10 with a pre-formed distal curve, the carrier tube 12 may bend along with and/or otherwise conform to the shape of the medical device 10.

In some instances, the carrier tube 12 may be substantially transparent. For example, the carrier tube 12 may be formed from a material (e.g., such as those disclosed herein) that allows for visualization of the medical device 10 within the carrier tube 12. In FIG. 1, the transparency of the carrier tube 12 is represented by the medical device 10 being shown in dashed/phantom line. For the purposes of this disclosure, this representation is intended to convey that the medical device 10 may be visible within the carrier tube 12.

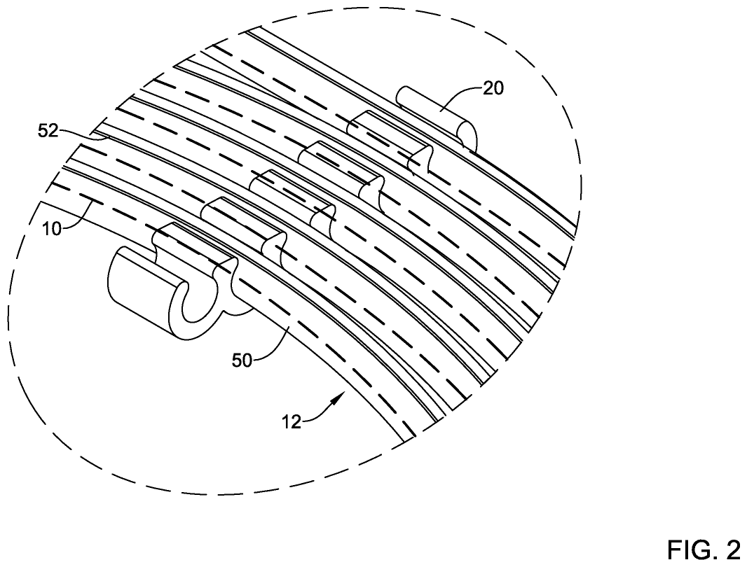

As shown in FIG. 2, the carrier tube 12 may have a cut or slit 52 formed therein (e.g., formed in the tube wall 48) as shown in FIG. 2. The slit 52 may be formed in the carrier tube 12 as a secondary operation (e.g., after the carrier tube 12 is formed). Alternatively, the slit 52 may be formed during an extrusion process using a suitable die configuration that forms the slit 52. The slit 52 may allow the medical device 10 to be efficiently loaded into the carrier tube 12 (e.g., as part of the packaging process) and/or allow the medical device 10 to be pull out of/removed from the carrier tube 12 prior to use. For example, to facilitate loading, the carrier tube may be pushed out slightly to widen the slit 52 and the medical device 10 can be inserted therein. This operation may utilize a loading tool that helps to widen/open the slit 52. After temporarily widening the slit 52, the elastomeric nature of the carrier tube 12 allows the slit 52 to shift back to its original shape/width. To remove the medical device 10, a clinician may simply pull upward on the medical device 10 through the slit 52.

The slit 52 may also help to hydrate/prepare the medical device 10 (e.g., to activate a hydrophilic coating) when such hydration steps are desired. For example, when hydration is desired, the carrier tube 12 can be disposed within or otherwise exposed to a hydrating fluid (e.g., saline) and the fluid may pass through the slit 52 into contact with the medical device 10. In addition, the slit 52 may help in sterilization of the medical device. For example, the slit 52 may allow for the relatively efficient passage of a sterilizing gas (e.g., ethylene oxide) therethrough.

In some instances, the slit 52 extends along the full length of carrier tube 12. In other instances, the slit 52 may extend along only a portion of the length of the carrier tube 12. For example, a relatively short distal section at the distal end 40 of the carrier tube 12 may lack the slit 52. In some instances, a plurality of slits 52 may be formed in the carrier tube 12. The plurality of slits 52 may be axially aligned or, alternatively, one or more of the slits 52 may be circumferentially offset from one another. In some instances, the slit 52 may be arranged along the “top” of the carrier tube 12. For example, when the carrier tube 12 is arranged in a spiral configuration (e.g., as depicted in FIG. 1) and the carrier tube 12 is laid flat and viewed from above, the slit 52 extends along a top surface of the carrier tube 12. This may allow a medical device (e.g., the medical device 10) to be efficiently loaded into and/or removed from the carrier tube 12. Other arrangements are contemplated for the slit 52.

With the medical device 10 disposed in the carrier tube 12, the carrier tube 12 may be disposed in an outer package/pouch. The outer package can be suitable sealed and undergo a suitable sterilization process.

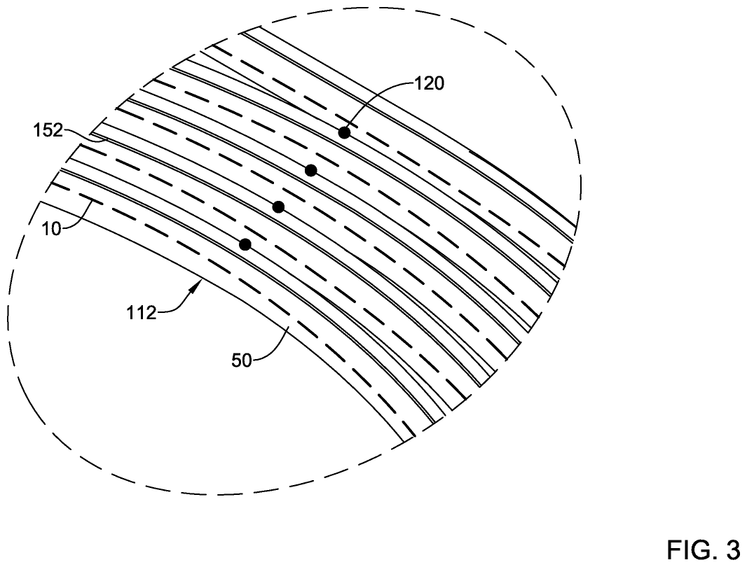

In some instances, the clips 20 may be used to secure adjacent windings of the carrier tube 12. However, this is not intended to be limiting. For example, FIG. 3 illustrates a carrier tube 112 where adjacent windings thereof are secured together with one or more discrete bonding members 120. In instances where the carrier tube 112 is formed form an elastomeric material such as silicone (and/or when the carrier tube 12 is formed from an elastomeric material), the carrier tube 112 may be sufficiently flexible and have a relatively low spring force so that the discrete bonding member 120 may be sufficient to secure together windings of the carrier tube 112. In some instances, the discrete bonding members 120 may include an adhesive. However, other discrete bonding members 120 are contemplated. For example, the one or more discrete bonding members 120 may include one or more thermal bonds (e.g., heat bonds). As shown in FIG. 3, the carrier tube 112 may also include a slit 152 formed therein.

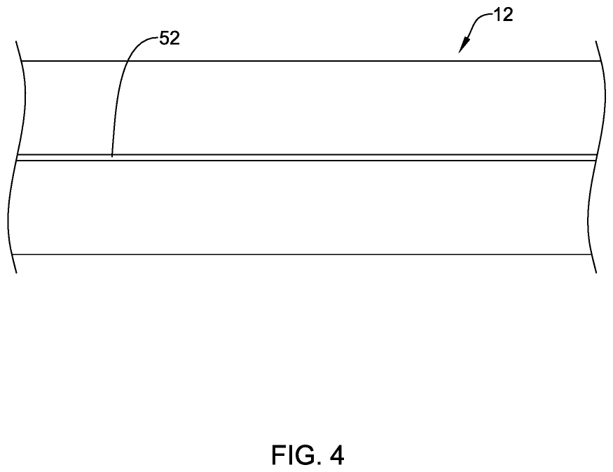

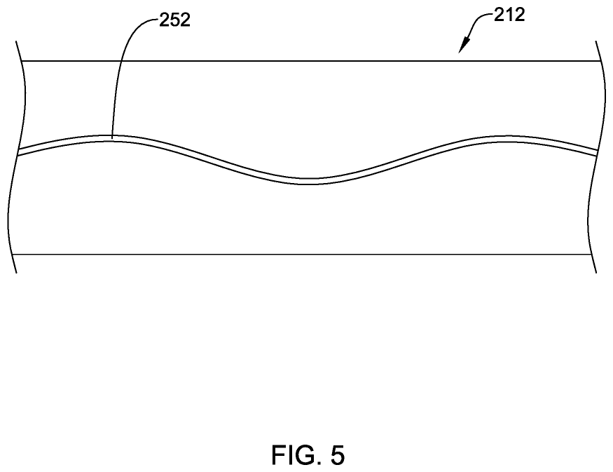

As disclosed herein, the carrier tubes disclosed herein (e.g., the carrier tubes 12, 112, as well as others) may include a slit 52, 152. In some instances, the slit 52 extends axially along the carrier tube 12 as depicted in FIG. 4. However, the slit 52 may have a different configuration. For example, FIG. 5 depicts a carrier tube 212 that includes an undulating slit 252 (e.g., a slit 252 with one or more curves/undulations). Such a configuration may help maintain the medical device 10 within the carrier tube 212 and may help to reduce the likelihood that the medical device 10 comes out of the carrier tube 212 unintentionally. Other slits are contemplated.

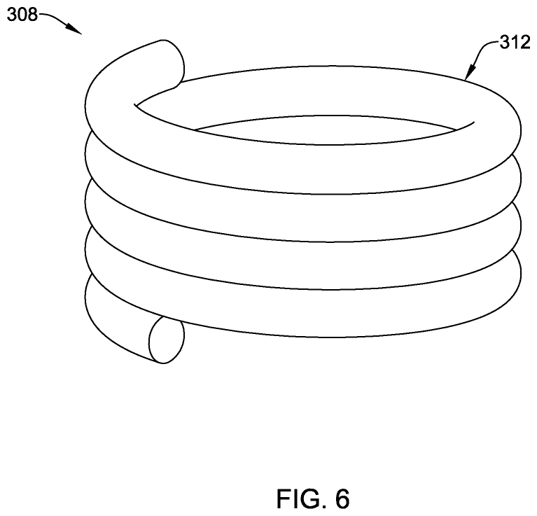

While FIG. 1 depicts that the carrier tube 12 may be arranged in a spiral configuration, other configurations are contemplated. For example, FIG. 6 depicts a medical device package 308 that includes a carrier tube 312 arranged in a helical configuration. The carrier tube 312, when arraigned in a helical configuration, may include a slit (not shown but may be similar to those disclosed herein). The slit may be arranged so that it extends along an outwardly facing surface of the carrier tube 312.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A package for a medical device, the package comprising:
an elongate carrier tube capable of having a medical device disposed therein;
wherein the carrier tube includes an elastomeric material; and
wherein the carrier tube includes a tube wall and wherein a slit is formed in the tube wall.

2. The package of claim 1, wherein the tube wall is substantially transparent so that the medical device can be visualized within the carrier tube.

3. The package of claim 1, wherein the elastomeric material includes silicone.

4. The package of claim 1, wherein the elastomeric material includes ethylene propylene diene monomer rubber, ethylene propylene rubber, polyethylene terephthalate glycol, polytetrafluoroethylene, foamed polytetrafluoroethylene, ethylene vinyl acetate, an ionomer, a fluoropolymer, or combinations thereof.

5. The package of claim 1, wherein the slit extends axially along the tube wall.

6. The package of claim 1, wherein the slit includes one or more undulations.

7. The package of claim 1, wherein the carrier tube is capable of be arranged in a spiral configuration.

8. The package of claim 7, wherein when the carrier tube is in the spiral configuration, the carrier tube includes a plurality of adjacent loops that are secured together by one or more discrete bonding members.

9. The package of claim 8, wherein at least some of the one or more discrete bonding members includes an adhesive.

10. The package of claim 1, wherein the carrier tube is capable of be arranged in a helical configuration.

11. A method for packaging a medical device, the method comprising:
extruding a carrier tube;
wherein the carrier tube includes an elastomeric material;
wherein the carrier tube includes a tube wall;
forming a slit in the tube wall; and
loading a medical device into the carrier tube by passing the medical device through the slit.

12. The method of claim 11, wherein the tube wall is substantially transparent, and further comprising inspecting the medical device by viewing the medical device through the tube wall.

13. The method of claim 11, wherein forming a slit in the tube wall includes forming an axial slit in the tube wall.

14. The method of claim 11, wherein forming a slit in the tube wall includes forming an undulating slit in the tube wall.

15. The method of claim 11, further comprising arranging the carrier tube in a spiral configuration.

16. The method of claim 15, wherein when the carrier tube is in the spiral configuration the carrier tube includes a plurality of adjacent loops, and further comprising securing together at least some of the plurality of adjacent loops with one or more discrete bonding members.

17. The method of claim 16, wherein at least some of the one or more discrete bonding members includes an adhesive.

18. The method of claim 11, further comprising arranging the carrier tube in a helical configuration.

19. A carrier tube for packaging a medical device, the carrier tube comprising:
an extruded silicone tube having a tube wall;
wherein the extruded silicone tube is substantially transparent; and
a slit formed in the tube wall.

20. The carrier tube of claim 19, wherein the slit includes an axial region, an undulating region, or combinations thereof.
